(19) United States
(12) Patent Application Publication
(10) Pub. No.: US 2009/0142318 A1
(43) Pub. Date: Jun. 4, 2009

(54) METHOD TO EXPAND nTREG CELLS USING p70 S6 KINASE ANTAGONIST

(75) Inventors: Steven Charles Eck, West Chester, PA (US); Li Li, Downingtown, PA (US)

Correspondence Address: HISCOCK & BARCLAY, LLP, 2000 HSBC PLAZA, 100 Chestnut Street, ROCHESTER, NY, 14604-2404, US

(73) Assignee: Therakos, Inc.

(21) Appl. No.: 12/364,628

(22) Filed: Feb. 3, 2009

Related U.S. Application Data
(63) Continuation-in-part of application No. 12/325,464, filed on Dec. 1, 2008.
(60) Provisional application No. 60/991,301, filed on Nov. 30, 2007.
(60) Provisional application No. 60/992,347, filed on Dec. 5, 2007.

(51) Int. Cl. A61K 35/12; C12N 5/06

(52) U.S. Cl. 424/93.71; 435/375

(57) ABSTRACT

Disclosed in this specification is a method to promote the growth of CD4+CD25Foxp3+ nTreg cells in a culture while treating the culture with a p70 S6 kinase inhibitor. The resulting cells are useful in the treatment of immune-related diseases.

### CROSS-REFERENCE TO RELATED APPLICATION

This application is a continuation-in-part of co-pending U.S. patent application Ser. No. 12/325,464 filed Dec. 1, 2008, which claims the benefit of U.S. provisional patent application Ser. No. 60/991,301, filed Nov. 30, 2007, and Ser. No. 60/992,347, filed Dec. 5, 2007, which applications are incorporated herein by reference in their entirety.

### FIELD OF THE INVENTION

This invention relates, in one embodiment, to a method for selectively growing nTreg cells preferentially over T effectors cells in the presence of a p70 S6 kinase inhibitor. The resulting nTreg cells are particularly useful for treating immune diseases, such as graft versus host disease.

### BACKGROUND OF THE INVENTION

T regulatory (Treg) cells are important in maintaining the homeostatic balance of the human immune system and immune tolerance. One of the most well studied types of Treg cells is the natural Treg (nTreg) cell CD4+ CD25+Foxp3+ cell. Defects in either the nTreg cells or in Foxp3 have been linked to unfavorable immune responses such as autoimmunity, allergic response, and organ rejection. Conversely, administration of healthy CD4+ CD25+Foxp3+ nTreg cells have demonstrated therapeutic effects in the treatment of a variety of animal disease models. Although the nTreg cells are a small fraction of the circulating lymphocyte pool it has been found that nTreg cells can be expanded ex vivo to provide clinically useful quantities of nTreg cells. The possibility therefore exists for using ex vivo expanded nTreg cells to regulate the immune response of a human being.

During the process, nTreg cells are withdrawn from peripheral blood mononuclear cells (PBMC) using magnetic bead-based methods. The enriched nTreg cells are activated with anti-CD3/CD28 coated beads in the presence of high concentrations (ca. 1000 U/ml) of human recombinant IL-2. Although the purified cells are enriched for nTreg using the bead-base methods, the resulting sample is not pure. Due to the lack of nTreg-specific surface markers, the sample almost always contains non-Treg cells that expressed similar cell surface markers (e.g. CD4 and CD25). After about three weeks of culture time, the nTreg cell populations underwent multiple folds of expansion. Typically under careful culture conditions, the expansion process involves a period of a few days in the first week where Foxp3 expression is close to, or even higher than that of the newly purified cells. This is followed by a period in which the percentage of cells expressing Foxp3 becomes smaller with continued cell expansion. The most likely explanation for the observed reduction of Foxp3 expressing cells is the outgrowth of cells which were Foxp3 negative at the cultures start. However, conversion of Foxp3 expressing cells to non-expressing cells in these cultures has not been ruled out. Careful culturing conditions are needed to prevent the non-nTreg cells from expanding faster than the nTreg cells and disturbing the overall composition of the sample. The overgrowth of non-Treg cells during Treg expansion not only potentially reduces the potency and effectiveness of the Treg cell therapy, but also provides a potential source of pro-inflammatory T effector cells and cytokines. Thus there is a need to find strategies and compounds to suppress the activation and growth of non-Treg cells in the cultured population.

It has been reported that rapamycin preferentially inhibits effector T cells over Treg cells, mostly likely through its activity on the mTOR complex. As such, rapamycin may be used to enhance the purity of nTreg cells that are cultured ex vivo. It would be advantageous to provide additional methods to inhibit T effectors cell expansion while permitting nTreg cell expansion.

### SUMMARY OF THE INVENTION

Applicants have discovered that p70 S6 kinase can be selectively inhibited to permit the growth of nTreg cells preferentially over T effector cells.

Disclosed in this specification is a method to selectively inhibit the growth of T effectors cells over nTreg cells using an antagonist of p70 S6 kinase. When cellular expansion is allowed to proceed in the presence of such an antagonist, an enriched population of nTreg cells is produced.

### DETAILED DESCRIPTION

P70 S6 kinase is part of a signaling pathway that includes mTOR. Without wishing to be bound to any particular theory, applicants believe that the effects of rapamycin on nTreg cells may be, at least in part, through the inhibition of p70 S6 kinase and that other p70 S6 kinase inhibitors may have beneficial effects similar to rapamycin. Since rapamycin lacks specificity it suffers from a certain degree of toxicity. If other inhibitors were available, a more specific (and therefore less toxic) alternative could be selected.

Using convention techniques CD4+ CD25+ T cells were purified from normal donor PBMC using standard Treg kits (Miltenyi) with AutoMacs. The purified cells were stained for Foxp3 and the percentage of Foxp3+ cells was determined using FACS. Approximately 50% of the purified CD4+ CD25+ cells were also Foxp3+ prior to expansion.

Purified CD4+ CD25+ cells were stimulated with anti-CD3/CD28 beads in the presence of IL-2 with various p70 S6 kinase inhibitors for two weeks as their population was allowed to undergo expansion. The expansion was allowed to continue for a sufficient period of time to permit a sizeable portion of cells to be obtained, but not for so long that unacceptable drift in the composition of the sample was realized. The expression of Foxp3 was determined using FACS.

TABLE 1

| Additive | % Foxp3+ |
|---|---|
| None | 21% |
| Rapamycin (100 nM) | 62% |
| 5,6-dichloro-1-beta-D- | 50% |
| ribofuranosylbenzimidazole (DRB) (12.5 mM) | |

As shown in Table 1, when no additive is used, the composition of the culture drifts to lower percentages of Foxp3+ cells. The most likely explanation of this observation is that the expansion of the Foxp3− cells begins to out-pace the expansion of the desired Foxp3+ cells. In the example given after two weeks, the composition of Foxp3+ cells had fallen to only 21%. The addition of 100 nM rapamycin caused the cellular composition to be increased in the percentage of Foxp3 expressing cells relative to its absence during the expansion process, presumably due to inhibition of mTOR. Applicants have discovered that p70 S6 kinase inhibits provide a benefit that is comparable with rapamycin. Inclusion of DRB in the culture medium consistently increased in the percentage of Foxp3 expressing cells relative to its absence. Other compounds with described P70 S6 inhibitory action were also tested to verify the relationship between Foxp3 expression and p70 S6 kinase inhibition.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A process for expanding the population of CD4+ CD25+ nTreg cells comprising the steps of:
enriching CD4+ CD25+ regulatory T cells by extracting the cells from a sample thus producing enriched CD4+ CD25+ regulatory T cells;
expanding the population of the enriched CD4+ CD25+ regulatory T cells while treating the enriched cells with a p70 S6 kinase inhibitor.

2. The process as recited in claim 1, wherein the p70 S6 kinase inhibitor includes 5,6-dichloro-1-beta-D-ribofuranosylbenzimidazole.

3. A process for reducing effects of graft versus host disease using ex vivo expanded CD4+ CD25+ regulatory T cells comprising the steps of:
obtaining a sample that includes peripheral blood mononuclear cells from a human donor, wherein the peripheral blood mononuclear cells includes CD4+ CD25+ regulatory T cells;
enriching the CD4+ CD25+ regulatory T cells in the sample thus producing enriched CD4+ CD25+ regulatory T cells;
expanding the population of the enriched CD4+ CD25+ regulatory T cells while treating the enriched cells with a p70 S6 kinase inhibitor; and
administering a portion of the expanded CD4+ CD25+ regulatory T cells to a human being to treat graft versus host disease.

4. The process as recited in claim 3, wherein the step of expanding the population is performed for at least one week, but less than three weeks.

5. The process as recited in claim 3, wherein the step of expanding the population is performed for at least five days, but less than four weeks.

6. The process as recited in claim 3, wherein the step of expanding the population is performed for about two weeks.

7. The process as recited in claim 3, wherein the step of enriching the CD4+ CD25+ regulatory T cells produces an enriched sample that is 40% to 80% CD4+ CD25+ regulatory T cells relative to the total cell population in the enriched sample.

9. The process as recited in claim 8, wherein, after the step of expanding the population, the sample is 40% to 78% CD4+ CD25+ regulatory T cells relative to the total cell population.

10. The process as recited in claim 3, wherein the concentration of the CD4+ CD25+ regulatory T cells in the sample, both before and after expansion, are equal within a range of about 10%.

11. The process as recited in claim 3, wherein the step of expanding the population is performed for a sufficient period of time to result in a fold change in cell population ranging from not less than 30 fold increase to not greater than 300 fold increase.

12. The process as recited in claim 3, wherein the p70 S6 kinase inhibitor includes 5,6-dichloro-1-beta-D-ribofuranosylbenzimidazole.
